(19) United States
(12) Patent Application Publication
(10) Pub. No.: US 2009/0196864 A1
(43) Pub. Date: Aug. 6, 2009

(54) INSECTICIDES THAT TARGET PROTEIN KINASE A (PKA)

(76) Inventor: Lee A. Bulla, Tioga, TX (US)

Correspondence Address: MORRISON & FOERSTER LLP, 12531 HIGH BLUFF DRIVE, SUITE 100, SAN DIEGO, CA, 92130-2040, US

(21) Appl. No.: 12/090,813

(22) PCT Filed: Oct. 20, 2006
(86) PCT No.: PCT/US06/41098

§ 371 Date: Jan. 9, 2009

Related U.S. Application Data
(60) Provisional application No. 60/728,532, filed on Oct. 20, 2005.

(51) Int. Cl. A01N 63/02; C12N 5/06; A01N 43/90; C12N 9/12; C07H 17/02; C12Q 1/48; C12Q 1/25

(52) U.S. Cl. 424/94.5; 435/348; 514/47; 435/194; 536/26.7; 435/15; 435/4

(57) ABSTRACT

The insecticides of the invention target the downstream portions of the signaling sequence of B. thuringiensis toxin, specifically protein kinase A (PKA) or adenylate cyclase (AC).

### CROSS-REFERENCE TO RELATED APPLICATIONS

This application is related to provisional application U.S. 60/728,532 filed 20 Oct. 2005. The contents of this document are incorporated herein by reference.

### TECHNICAL FIELD

The invention relates to the design and composition of insecticides that kill insects by targeting and activating protein kinase A (PKA) and/or adenylate cyclase (AC). These enzymes are responsible for a cascade of downstream factors that dismantle the cell in which it is located.

### BACKGROUND ART

It has been recognized for some time that intracellular signaling pathways offer a multiplicity of target sites for intervention in attempts to ameliorate disease conditions or to induce apoptosis. Thus, although a receptor for a particular cell-stimulating agent may mediate the downstream consequences of interaction with a ligand, these consequences can alternatively be affected by intervening in the sequence downstream of the initial signal. This was pointed out recently, for example, in an article by Fishman, M. C. et al., Nature (2005) 437:491-493.

The present invention reflects an application of this approach to insecticide formulation. As shown herein, activation of protein kinase A (PKA) is a downstream consequence of interaction of monomeric Cry1Ab toxin of B. thuringiensis with its cognate receptor in H5 cells (ovarian cells of the cabbage looper Trichoplusia ni) modified to contain an expression system for the receptor BT-R₁ (GenBank: AF319973). Activation of PKA can be achieved, however independent of interaction of the receptor with its ligand. Thus, it is possible to effect cell death by activating PKA directly, or by activating the upstream adenylate cyclase (AC).

In an earlier publication, Zhang, X. et al., Cell Death and Differentiation (2005) 12:1407-1416, the present applicants demonstrate that Cry1Ab toxin induces cell death only in the presence of the cognate receptor and that a magnesium ion dependent signaling pathway is required for cytotoxic action.

To summarize briefly, in a cell-based system, BT-R₁, which originally was isolated from midgut epithelium of the tobacco hornworm Manduca sexta, was heterologously expressed in High Five (H5) cells originated from ovarian cells of the cabbage looper, Trichoplusia ni. Cultured H5 cells were not affected by the toxin because they do not express any receptor for the toxin. Heterologous expression of BT-R₁, rendered transfected cells (S5) susceptible to the Cry1Ab toxin. Toxin-treated S5 cells exhibited dramatic morphological changes including altered size, shape and overall appearance whereas untreated viable cells remained compact and uniformly round. Time-lapse microscopy showed that S5 cells underwent sequential cytological changes upon toxin exposure. Cytotoxicity and cell death involved two rather distinct sequential events or stages: (i) membrane blebbing that occurred within 20 minutes after toxin exposure and (ii) cell swelling and lysis within 40 min, apparently the result of increased membrane permeability.

The morphological changes observed in Cry1Ab toxin-treated cells are strikingly similar to the phenotypic changes associated with oncosis. Osmotic protectants such as glucose, sucrose and raffinose can counter osmotic pressure produced by a drastic ion flux across cell membrane. The protectants prevent cell swelling only when their molecular size is larger than the active ion channels in the cell membrane. The molecular diameters of raffinose, sucrose and glucose are approximately 1.2-1.4 nm, 0.9 nm and 0.7 nm, respectively. In the presence of raffinose (30 mM), toxin-exposed cells were arrested in the blebbing stage and did not swell. Sucrose (30 mM) partially prevented cell swelling whereas glucose (30 mM) did not interfere with either cell blebbing or swelling. Although raffinose-treated S5 cells did not undergo swelling after exposure to the Cry1Ab toxin, these cells eventually died, indicating that cytotoxicity is related to certain cellular events upstream of the swelling stage.

Cytotoxicity was also tested in the presence of the divalent cation chelators EGTA and EDTA by Trypan blue exclusion analysis as described earlier and by monitoring morphological changes of cells undergoing cell death. EDTA, not EGTA, completely abolished toxin-induced death in S5 cells (upper and lower center panels, respectively). Cells pre-treated with EGTA were fully susceptible to Cry1Ab toxin. On the other hand, EDTA-treated cells were refractory to Cry1Ab toxicity, but addition of Mg²⁺ (5 mM) to EDTA-treated cells restored toxin-mediated lethality whereas Ca²⁺ (5 mM) had no such effect. Removal of Mg²⁺ by EDTA or inhibition of toxin binding to BT-R₁, prevents S5 cells from blebbing and inhibits cell death completely. Evidently, a Mg²⁺-dependent intracellular pathway is established upstream of the blebbing stage (within 5 min after toxin binding to BT-R₁, on S5 cells) and is critical to toxin-induced cell death.

### DISCLOSURE OF THE INVENTION

In one aspect, the invention is directed to a method to kill insect cells by providing said cells with an activator of PKA or of adenylate cyclase (AC). Activators of PK may include cyclic AMP provided the appropriate association with the target is insured. Other activators of PKA and of AC are known in the art, as set forth below.

In another aspect, the invention provides compositions that are lethal to insects which contain PKA or AC activators. The insects that are subject to the insecticidal methods and compositions of the invention are of any variety, especially those that are subject to the insecticidal action of B. thuringiensis toxin. B. thuringiensis is toxic to a wide variety of insect classes, including Leipidoptera and Trichoplusia.

In another aspect, the invention is directed to a method to identify a compound or protocol that is insecticidal by evaluating the effect of the compound or protocol on the activity of PKA or AC. Substances and protocols that activate PKA or AC are thus identified as candidate insecticides.

Assays for evaluation of PKA activation are well known in the art. PKA phosphorylates a number of proteins, and the phosphorylation of these proteins serves as an index of PKA activity. Assays for evaluation of AC activation are also well-known as cyclic AMP, the product, can be assayed, including by its effect on phosphorylation events catalyzed by PKA.

### BRIEF DESCRIPTION OF THE DRAWINGS

FIG. 1. Proposed model for the action of Cry toxins. According to the model, toxin binds to the receptor and stimulates the production of intracellular cAMP, which in turn activates a cAMP-PKA signal pathway leading to oncotic cell death. Activation of this cAMP-PKA pathway involves in G proteins and adenylate cyclase.

FIG. 2. Effects of various chemical treatments on Cry1Ab toxin-induced cytotoxicity and cAMP levels in S5 cells. (a) Cells were incubated with IBMX (0.5 mM) before the addition of Cry1Ab and CAMP was measured by determining the intensity of fluorescence in High Five cells (3×10⁴). (b) S5 cells were incubated for 30 min with ddADP (40 mM), a cell-permeable inhibitor of adenylate cyclase prior to the addition of Cry1Ab toxin (60 nM). Cytotoxicity was determined by Trypan blue exclusion analysis. (c) Percent relative cytotoxicity of Cry1Ab in the presence of NF023 (1 μM) and NF449 (1 μM). (d) cAMP levels in High Five cells after treatment with Cry1Ab alone (Cry, 180 nM) or in the presence of EDTA (5 mM) (EDTA+Cry) for 10 min, forskolin (FSK, 2 μM) or the combination of EDTA and forskolin and (EDTA+FSK). (e) Percent relative cytotoxicity of Cry1Ab in the presence of DDA2P (40 μM), pCPT-cAMP (200 μM) and Forskolin (FSK, 2 μM). (f) Percent relative cytotoxicity of Cry1Ab in the presence of cell-permeable inhibitors of PKA (H-89, 0˜50 μM and PKAI 14-22-Amide, 0˜8 μM) for 30 min at the specified concentration before addition of Cry1Ab toxin (180 nM). Data are presented as the mean±S.D. of six experiments.

### MODES OF CARRYING OUT THE INVENTION

Execution of cell death appears to involve a relatively limited number of evolutionarily conserved mechanisms connected to cell signaling pathways that lie inactive. Activation of such pathways is tightly regulated since they govern cellular activities critical to survival and cell fate. Unusual activity and disruption of cell signaling pathways can cause deregulation in cells, alter gene expression and bring structural and functional disarray resulting in abnormal and uncontrolled cell division or cell death. Many pathogenic organisms and their toxins target host cell receptors causing peculiarity in cell signaling events that alters cellular processes or leads to cell death. There is a significant body of literature that describes various molecular and cellular aspects of toxins associated with bacterial invasion, colonization and host cell disruption. However, there is little information on the molecular and cellular mechanisms associated with the insecticidal action of Bacillus thuringiensis (Bt) Cry toxins.

One BT receptor, BT-R₁, represents a family of insect cadherins that are expressed in the midgut of lepidopteran larvae during development. Lethal action of the toxin in susceptible insect larvae is mediated by high-affinity binding of the toxin to the membrane-proximal ectodomain of the cadherin receptor leading to destruction of the midgut epithelium and death of insect.

It has now been found that binding of Cry toxin to BT-R₁, provokes oncotic cell death in insect cells by activating a signaling pathway involving stimulation of G protein and adenylate cyclase (AC), increased cAMP levels and activation of PKA. Activation of a cAMP/PKA signaling pathway by Cry toxin only in BT-R₁-expressing insect cells indicates that oncosis involves assembly of a unique cell surface receptor-mediated pathway of cell death.

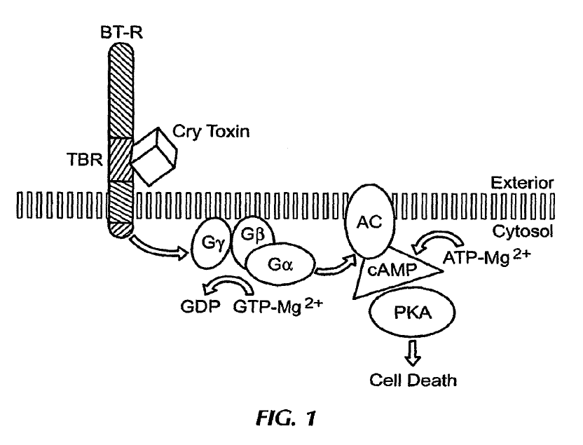

FIG. 1 represents the pathway of effecting cell death by interaction with BT-R₁, as verified by the examples herein. As shown, interaction of the Cry toxin with the appropriate receptor activates a magnesium ion dependent G protein which in turn activates adenylate cyclase (AC) producing cyclic AMP which in turn activates PKA. PKA directly leads to cell death. Accordingly, compounds that activate either adenylate cyclase (AC) or PKA can activate this pathway and ultimately effect cell death. Activators of such enzymes are well-known in the art.

For instance, commercially available PKA inhibitors include adenosine-3′,5′-cyclic monophosphorothioate, Sp-isomer (Sp-cAMPS); adenosine-3′,5′-cyclic monophosphorothioate, acetoxymethyl ester, Sp-isomer (Sp-cAMPS-AM); 8-bromoadenosine-3′,5′-cyclic monophosphorothioate, Sp-isomer (Sp-8-Br-cAMPS); 8-bromoadenosine-3′,5′-cyclic monophosphorothioate acetoxymethyl ester, Sp-isomer (Sp-8-Br-cAMPS-AM); 8-chloroadenosine-3′,5′-cyclic monophosphorothioate, Sp-isomer (Sp-8-Cl-cAMPS); 8-(4-chlorophenylthio) adenosine-3′,5′-cyclic monophosphorothioate, Sp-isomer (Sp-8-CPT-cAMPS); 5,6-dichloro-1-13-D-ribofuranosylbenzimidazole-3′,5′-cyclic monophosphorothioate, Sp-isomer (Sp-5,6-DCl-cBIMPS); 8-hydroxyadenosine-3′,5′-cyclic monophosphorothioate, Sp-isomer (Sp-8-OH-cAMPS); 2′-O— monobutyryladenosine-3′,5′-cyclic monophosphorothioate, Sp-isomer (Sp-2′-O-MB-cAMPS); 8-piperidinoadenosine-3′,5′-cyclic monophosphorothioate, Sp-isomer (Sp-8-PIP-cAMPS); 8-(2-aminoethylamino)adenosine-3′, 5′-cyclic monophosphorothioate, Sp-isomer (Sp-8-AEA-cAMPS); 8-azidoadenosine-3′, 5′-cyclic monophosphorothioate, Sp-isomer (Sp-8-N3_cAMPS); 2-chloroadenosine-3′, 5′-cyclic monophosphorothioate, Sp-isomer (Sp-2-Cl-cAMPS); 6-chloropurine riboside-3′, 5′-cyclic monophosphorothioate, Sp-isomer (Sp-6-Cl-cPuMPS); 7-deazaadenosine-3′, 5′-cyclic monophosphorothioate, Sp-isomer (Sp-7-CH-cAMPS); 6-dimethylaminopurine riboside-3′,5′-cyclic monophosphorothioate, Sp-isomer (Sp-6-DMA-cAMPS); 6-ethylthiopurine riboside-3′,5′-cyclic monophosphorothioate, Sp-isomer (Sp-6-EtS-cAMPS); inosine-3′,5′-cyclic monophosphorothioate, Sp-isomer (Sp-cIMPS); 8-iodoadenosine-3′, 5′-cyclic monophosphorothioate, Sp-isomer (Sp-8-1-cAMPS); N⁶-phenyladenosine-3′, 5′-cyclic monophosphorothioate, Sp-isomer (Sp-6-Phe-cAMPS); and purine riboside-3′, 5′-cyclic monophosphorothioate, Sp-isomer (Sp-cPuMPS).

Suitable AC inhibitors that are readily available include adenylate cyclase toxin, Bordetella pertussis; Recombinant, E. coli; cholera toxin, A Subunit, Vibrio cholerae, Type Inaba 569B; cholera toxin, Vibrio cholerae, Type Inaba 569B; cholera toxin, Vibrio cholerae, Type Inaba 569B, Azide Free; L-(−)-epinephrine-(+)-bitartrate; forskolin, 1,9-dideoxy-, Coleus forskohlii; forskolin, 7-deacetyl-7-[O-(N-methylpiperazino)-γ-butyryl]-, Dihydrochloride; forskolin, 7-deacetyl-7-o-hemisuccinyl-; forskolin, Coleus forskohlii; isoproterenol hydrochloride; PACAP 27 amide, ovine; and PACAP 38, ovine.

The insecticide compositions containing these inhibitors can be prepared using formulation methods known in the art. The formulations can be applied to aqueous environments or included in feeding preparations intended for the insect targets. Alternatively, the formulation may penetrate the cells upon topical application.

The example that follows provides the basis for the present invention.

### Example 1

### Involvement of AC and PKA in Oncotic Insect Cell Death

A number of bacterial toxins kill target cells by receptor-mediated endocytosis and disruption of essential cytosolic functions. Some pathogenic bacteria also produce pore-forming toxins or protein synthesis inhibitors that are associated with apoptosis of the target cells.

First, endocytosis was excluded as a mechanism. S5 cells, Zhang, et al., supra, were treated with endocytosis inhibitors prior to toxin addition. These cells underwent the same morphological changes as those cells not exposed to the inhibitors (see Table 1 below). Furthermore, none of the inhibitors precluded cell death as indicated by Trypan blue exclusion analysis (data not shown). As was shown previously by fluorescence microscopy, the toxin is not internalized at either stage of cytotoxicity.

Apoptosis was also excluded as a mechanism of effecting cell death in the Cry1 Ab toxin pathway. The broad-spectrum caspase inhibitor z-VAD-fmk, which prevents apoptosis in many cell types, did not suppress Cry1Ab-induced toxicity and cell death. Likewise, the serine protease inhibitors Pefabloc and TPCK and the cathepsin inhibitor E-64 had no effect (see Table 1). The S5 cells treated with all of the inhibitors underwent cell death upon exposure to Cry1Ab in a manner similar to cells exposed to toxin alone. In addition, no externalization of phosphatidylserine or DNA fragmentation was observed in toxin-treated cells (data not shown). Evidently, the Cry1Ab toxin of Bt does not cause insect cell death with features of either endocytosis or apoptosis. Rather, cell death is oncotic.

TABLE 1
Characterization of toxin-induced cell death

| Inhibitor | Cellular targets | Effect(s) | Cell death¹ |
|---|---|---|---|
| Control | n.a.² | n.a. | + |
| Nocodazole | Microtubule | Endocytosis | + |
| Cytochalasin D | Actin | Endocytosis | + |
| Phenylarsine oxide | n.d.³ | Endocytosis | + |
| Bafilomycin A | H⁺-ATPases | Endocytosis | + |
| Z-VAD-fmk | Caspases | Apoptosis | + |
| Pefabloc | Serine proteases | Apoptosis | + |
| TPCK | Serine proteases | Apoptosis | + |
| E-64 | cathespins | Apoptosis | + |
| EGTA | Metalloproteins | Ca²⁺ | + |
| EDTA | Metalloproteins | Mg²⁺, Ca²⁺ | − |

¹Cells were switched to inhibitor-free buffer before Cry1Ab toxin treatment. Cell death is the percent of dead cells in the total population as determined by Trypan blue staining of nuclei; + = 80 ± 5% cell death; − = no cell death. Data are the mean ± S.D. of six experiments.
²n.a., not applicable.
³n.d., not determined.

Cell death was prevented completely upon removal of Mg²⁺ by EDTA (Table 1). As noted above, that removal of Mg²⁺ does not impede toxin binding to BT-R₁, indicating that Mg²⁺ is required for toxicity downstream to receptor binding. (Zhang, et al., supra) Because Mg²⁺ is a key component in cytotoxicity and because it allows progressive cell death, it was suggested that adenylate cyclase and heterotrimeric GTP-binding protein, both of which require Mg²⁺, are involved in a signaling pathway induced by the binding of toxin to BT-R₁. (See FIG. 1)

Adenylate cyclase (AC) is an integral membrane protein and catalyzes the conversion of ATP to cyclic AMP (cAMP) in a Mg²⁺-requiring reaction. The activity of membrane-bound AC is regulated by heterotrimeric G proteins, the largest group of cell surface proteins involved in signal transduction. The stimulatory G protein alpha-subunit (G_{αs}) dissociates from the P and γ subunits through a Mg²⁺-dependent GTP exchange reaction and binds to the catalytic part of the AC, inducing a structural change that stimulates enzyme activity and cAMP production. Cyclic AMP functions as a “second messenger” to relay extracellular signals to intracellular effectors such as cAMP-dependent protein kinase (PKA). Cyclic AMP-activated PKA phosphorylates specific substrate proteins. In the phosphorylated form, these protein substrates become active and participate in a broad range of biological activities such as cellular response to various stimuli, including bacterial toxins. Thus, Cry1Ab toxin binding to the cadherin receptor BT-R₁, provokes a G protein coupled cAMP/PKA signaling pathway that, upon activation, causes abrupt changes in the cytoskeleton and brings about aberrant ion channel activity leading to cell death, as shown in FIG. 1.

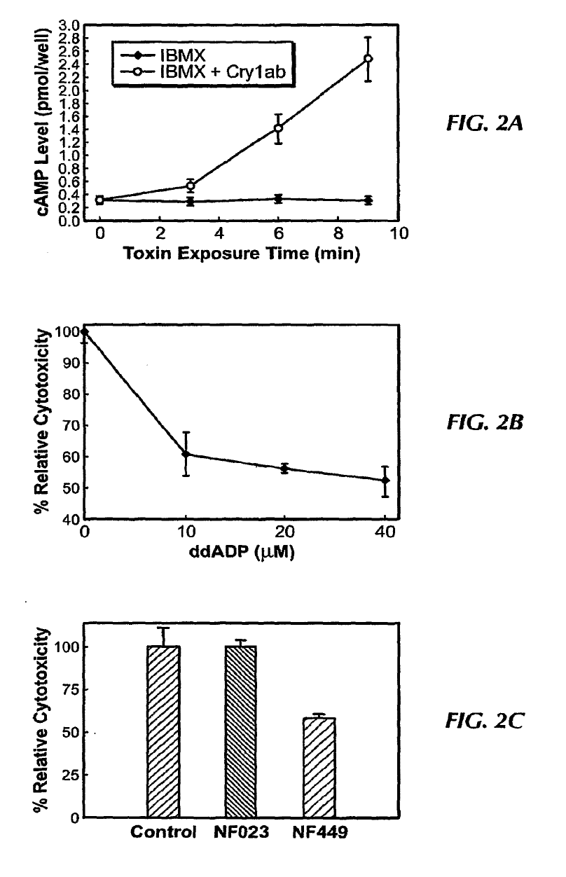

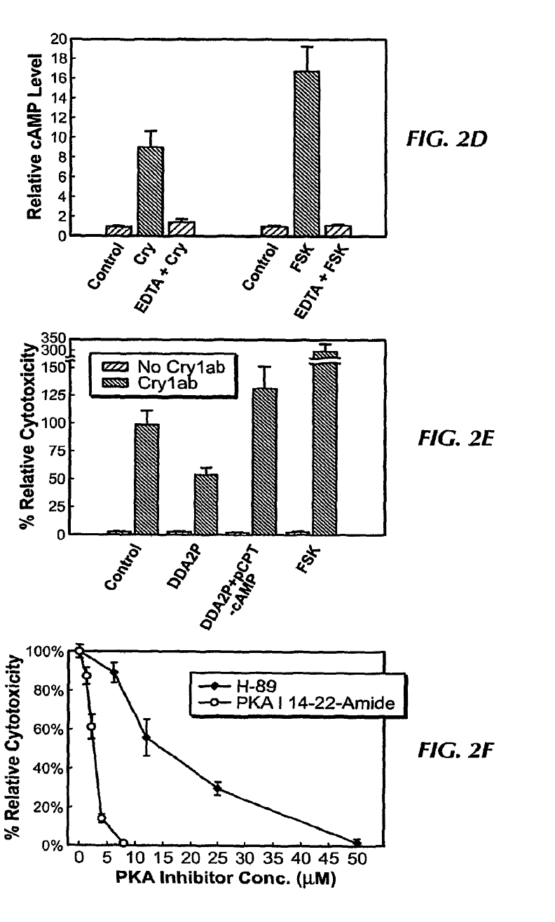

To verify this, intracellular cAMP production was measured in toxin-treated S5 cells over time. In the presence of the phosphodiesterase inhibitor IBMX, cAMP production increased in a time-dependent manner in these toxin treated cells (FIG. 2a). It is known that IBMX prevents cAMP degradation and facilitates the accumulation of detectable levels of cAMP in the S5 cells. Incubation of the cells with IBMX in the absence of the Cry1Ab toxin did not cause any elevation in the level of cAMP (FIG. 2a), and cAMP levels did not change in receptor-free H5 cells (data not shown), substantiating that Cry toxin binding to BT-R₁ is critical in stimulating cAMP production.

In addition, 2′,5′-dideoxy-3′-ADP (ddADP), which interferes with AC activity by blocking substrate utilization by the enzyme, prevented Cry1Ab-induced cytotoxicity and cell death. As can be seen in FIG. 2b, ddADP interfered with Cry1Ab toxicity in a dose-dependent manner, significantly reducing the number of cells undergoing cell death (50% less). That ddADP did not completely eliminate the toxic effects of Cry1Ab most likely is because ddADP is not fully inhibitory to AC in the S5 cells. Nevertheless, a signaling pathway involving AC and the second messenger cAMP is activated by Cry toxin.

Membrane-bound AC is activated by the stimulatory G protein alpha subunit (G_{αs}). To ascertain whether a G protein switch is turned on in a Cry toxin-induced pathway, a cell-permeable inhibitor, NF449, Hohenegger, M. et al., Proc. Natl. Acad. Sci. (1998) U.S.A 95:346-351 (1998), which selectively antagonizes G_{αs} is used. S5 cells pre-incubated (30 min) with NF449 (1 μM) were less sensitive to the Cry1Ab toxin than untreated cells (FIG. 2c). The relative toxicity, as determined by cell death, mediated by Cry1Ab decreased 50 percent when S5 cells were incubated with NF449, indicating that G_{αs} participates in Cry1Ab toxicity by activating AC.

Another G-protein antagonist for α-subunits of the Gₒ/Gᵢ group, NF023, did not have any effect on toxicity when used at the same concentration (1 μM) as NF449 (FIG. 2c). NF449 did not completely retard Cry1Ab toxicity and cell death, suggesting that NF449 does not fully antagonize G_{αs} in S5 cells. Alternatively, a separate event downstream to Cry1Ab binding to BT-R₁, and not-involving G_{αs} might be involved in toxicity because ddADP also did not fully block the toxicity exerted by Cry1Ab (FIG. 2b). Nevertheless, activation of G protein and stimulation of AC leading to an increase in production of the second messenger cAMP appears to be associated directly with toxicity and cell death.

Because Mg²⁺ is required not only for G_{αs} activation through GTP exchange and subunit association but also for ATP binding and catalytic synthesis of cAMP, the effect of EDTA on Cry1Ab-induced cAMP levels in S5 cells was tested. Pre-treatment of S5 cells with EDTA (5 mM) virtually precluded any increase in cAMP levels (FIG. 2d) and prevented cell death when cells were exposed to Cry1Ab (results not shown). Furthermore, when S5 cells were pre-treated with the AC stimulator forskolin (FSK, 2 μM), intracellular cAMP levels were dramatically increased (FIG. 2d) and toxicity of Cry1Ab likewise was pronounced (FIG. 2e). EDTA precluded FSK induction of cAMP to the same extent as it did in cells not incubated with FSK (FIG. 2d). When S5 cells were pre-treated with the cAMP analog pCPT-cAMP (200 μM), even in the presence of the AC inhibitor ddADP (40 μM), toxicity of Cry1Ab was significantly increased (almost 100% cell death), relative to toxin-treated cells without ddADP (FIG. 2e).

These results clearly demonstrate that Cry toxin-induced toxicity and cell death accompany cAMP production. Based on this finding, stimulation of intracellular cAMP production by FSK was tested for its ability to induce cell death in S5 cells without Cry1Ab treatment. Neither FSK nor pCPT-cAMP alone was sufficient to bring about cell death in this experiment (FIG. 2e), indicating that elevation of intracellular cAMP is not sufficient but is required to mediate cytotoxicity when cells are under Cry toxin stress. These results suggest that an alternative pathway may permissively effect activation and/or progression of oncotic cell death.

Typically, cAMP activates cAMP-dependent PKA, a serine/threonine protein kinase. In the presence of cAMP, the catalytic subunit of PKA dissociates from the regulatory subunit/holoenzyme complex and phosphorylates various cellular proteins, including cytoskeletal proteins and ion channels, among others, and alters cellular activity. To determine whether the toxicity of Cry1Ab is mediated by a cAMP/PKA signaling event, the effects of two potent cell-permeable PKA inhibitors, H-89 and myristoylated amide 14-22 were tested. H-89 is a competitive inhibitor that interferes with ATP utilization by PKA, whereas amide 14-22 is a peptide substrate inhibitor. The PKA inhibitors were introduced to S5 cells in a pre-incubation step followed by addition of Cry1Ab toxin. Both inhibitors prevented Cry toxin-induced cell death in cultured S5 cells in a dose-dependent manner (FIG. 2f). Fifty μM H-89 fully blocked cell death. Similarly, 8 μM amide 14-22 prevented cell death indicating that inhibition of PKA abolishes Cry toxin action. Significantly, the characteristic morphological changes, including membrane blebbing and cellular swelling along with cell death, were completely prevented by PKA inhibition (results not shown), demonstrating that cAMP-dependent PKA is cardinal in Cry toxin action.

In light of these findings, it is clear that activation of a cAMP/PKA signaling pathway results in insect cell death.

1. A method to exert a cytotoxic effect on insect cells which method comprises contacting said cells with at least one substance that effects the activation of protein kinase A (PKA) or adenylate cyclase (AC).

2. The method of claim 1 wherein the insect cells are susceptible to the cytotoxic action of B. thuringiensis toxin.

3. The method of claim 1 wherein the cells are contained in an intact insect.

4. A composition comprising an effective amount of an active ingredient toxic to insect cells, wherein said active ingredient is an activator of PKA or AC.

5. A method to identify a compound or protocol that is effective as an insecticide, which method comprises assessing the ability of a substance or protocol to activate PKA or AC, wherein a substance or protocol that activates PKA or AC is identified as a candidate insecticide.
